(19) United States
(12) Patent Application Publication
(10) Pub. No.: US 2008/0045486 A1
(43) Pub. Date: Feb. 21, 2008

(54) OCULAR ADMINISTRATION OF TESTOSTERONE

(76) Inventors: Diane D.S. Tang-Liu, Newport Beach, CA (US); Zhiling Yu, Monarch Beach, CA (US); Ronald Bradford, Mission Viejo, CA (US)

Correspondence Address: ALLERGAN, INC., 2525 DUPONT DRIVE, T2-7H, IRVINE, CA, 92612-1599, US

(21) Appl. No.: 11/465,346

(22) Filed: Aug. 17, 2006

(51) Int. Cl. A61K 31/57

(52) U.S. Cl. 514/170; 514/177

(57) ABSTRACT

The present invention provides improving health in a woman or man comprising non-orally administering a testosterone-containing moiety to said woman or man by topically applying a liquid composition including said testosterone-containing moiety to the eye of said woman or man in an amount sufficient to provide a therapeutic effect.

### BACKGROUND OF THE INVENTION

1. The Field of the Invention

This invention relates to the ophthalmical administration of androgens e.g. testosterone, to provide hormone replacement therapy to women and men. Thus, this invention covers the fields of pharmaceutical sciences and medicine.

2. Background of the Art

Endogenous androgens, including testosterone and dihydrotestosterone (DHT), are responsible for the normal growth and development of the male sex organs and for maintenance of secondary sex characteristics. These effects include the growth and maturation of prostate, seminal vesicles, penis, and scrotum; the development of male hair distribution, such as facial, pubic, chest, and axillary hair; laryngeal enlargement, vocal chord thickening, alterations in body musculature, and fat distribution. Testosterone and DHT are necessary for the normal development of secondary sex characteristics. Male hypogonadism results from insufficient secretion of testosterone and is characterized by low serum testosterone concentrations. Symptoms associated with male hypogonadism include impotence and decreased sexual desire, fatigue and loss of energy, mood depression, regression of secondary sexual characteristics and osteoporosis. Hypogonadism is a risk factor for osteoporosis in men.

During exogenous administration of androgens, endogenous testosterone release may be inhibited through feedback inhibition of pituitary luteinizing hormone (LH). At large doses of exogenous androgens, spermatogenesis may also be suppressed through feedback inhibition of pituitary follicle-stimulating hormone (FSH).

It is known that a functional level of androgenic hormones such as testosterone in females promotes sexual health and activity, feelings of well being, maximizes muscle mass and function, and inhibits bone loss. Furthermore, a functional level of androgenic hormones may promote cardiovascular and coronary health, decrease breast tenderness, decrease vasomotor instability, modulate immune function, enhance certain cognitive abilities, improve urogential health, reduce estrogen supplementation related side effects, and provide direct neuroprotective effects.

The normal range of total serum testosterone concentrations in males is 300 to 1100 ng/dL. Endogenous total serum testosterone concentrations in normal males follow a diurnal pattern, but young and old men have different patterns. In young men, mean concentrations of total serum testosterone range from a peak of about 760 ng/dL in the morning to a trough of about 520 ng/dL in the evening, and generally decline during the day. In old men, mean concentrations of total serum testosterone are more constant and range from about 450 ng/dL to 550 ng/dL throughout the day (Wilson, 1996).

The daily rate of testosterone production in ovulating women is 0.4±0.1 mg (Vierhapper et al, 1997), which is about one-tenth the daily production in men. About half of this is derived from the metabolic conversion of androstenedione to testosterone at extraglandular sites (Wilson, 1996).

Alterations in plasma concentrations of testosterone and androstenedione occur during the menstrual cycle (Wilson, 1996). Different references list slightly different but overlapping ranges of normal values in women. The concentration of testosterone in the plasma of women ranges from 15 to 65 ng/dL. The normal laboratory range of total testosterone in adult (but not necessarily postmenopausal) females at Massachusetts General Hospital is 25-90 ng/dL (Wilson, 1996). The average plasma concentration of testosterone in ovulatory women is 40 ng/dL, but this falls by nearly 50% after menopause to an average of ˜24 ng/dL (Carr et al, 1998.).

The most extensive study of testosterone concentrations after menopause was conducted over a 5 year period in 4,040 healthy postmenopausal women ranging from 40 to 69 years old, and found that the mean concentration of total testosterone in this population the morning after an overnight fast was 25 ng/dL(Berrino et al, 1996).

A diurnal variation in plasma or serum testosterone concentrations in postmenopausal women is either nonexistent (Lisse et al, 1980; Strickler et al, 1981) or exists but is statistically insignificant and is of a moderate magnitude compared to variations from other causes (Lonning et al, 1989).

The following testosterone delivery systems are officially approved in USA by the Food and Drug Administration for hormone replacement in men:

ANDROGEL® (Unimed) (testosterone gel) 1% Rx only

AndroGel is indicated for replacement therapy in males for conditions associated with a deficiency or absence of endogenous testosterone. AndroGel (testosterone gel) delivers physiologic amounts of testosterone, producing circulating testosterone concentrations that approximate normal levels (298-1043 ng/dL) seen in healthy men.

AndroGel® (testosterone gel) 1% is a clear, colorless hydroalcoholic gel containing 1% testosterone. AndroGel provides continuous transdermal delivery of testosterone, the primary circulating endogenous androgen, for 24 hours following a single application to intact, clean, dry skin of the shoulders, upper arms and/or abdomen. A daily application of AndroGel 5 g, 7.5 g, or 10 g contains 50 mg, 75 mg, or 100 mg of testosterone, respectively, to be applied daily to the skin's surface. Approximately 10% of the applied testosterone dose is absorbed across skin of average permeability during a 24-hour period.

TESTIM® 1% (Auxilium) (testosterone gel) Rx only

Testim® is indicated for testosterone replacement therapy in adult males for conditions associated with a deficiency or absence of endogenous testosterone. Testim® 1% (testosterone gel) delivers physiologic amounts of testosterone, producing circulating testosterone levels that approximate normal levels (e.g., 300-1000 ng/dL) seen in healthy men.

Testim® (testosterone gel) is a clear to translucent hydroalcoholic topical gel containing 1% testosterone. Testim® provides continuous transdermal delivery of testosterone for 24 hours, following a single application to intact, clean, dry skin of the shoulders and upper arms. One 5 g or two 5 g tubes of Testim® contains 50 mg or 100 mg of testosterone, respectively, to be applied daily to the skin's surface. Approximately 10% of the applied testosterone dose is absorbed across skin of average permeability during a 24-hour period.

STRIANT® (Columbia) (testosterone buccal system) mucoadhesive

Striant® is indicated for replacement therapy in males for conditions associated with a deficiency or absence of endogenous testosterone. Striant® delivers physiologic amounts of testosterone to the systemic circulation, thereby producing circulating testosterone concentrations in hypogonadal males that approximate physiologic levels seen in healthy young men (300-1050 ng/dL).

Striant® (testosterone buccal system) is designed to adhere to the gum or inner cheek. It provides a controlled and sustained release of testosterone through the buccal mucosa as the buccal system gradually hydrates. Insertion of Striant® twice a day, in the morning and in the evening, provides continuous systemic delivery of testosterone.

Striant® is a white to off-white colored, monoconvex, tablet-like, mucoadhesive buccal system. Striant® adheres to the gum tissue above the incisors, with the flat surface facing the cheek mucosa. The active ingredient in Striant® is testosterone. Each buccal system contains 30 mg of testosterone.

Currently no form of testosterone therapy is officially approved for women in USA by the Food and Drug Administration. However for many years testosterone has been in used in public hospital specialist clinics, and in private practices for postmenopausal women with low testosterone levels. Decisions on use need to be made in partnership between women and their doctor.

The present invention provides a new delivery method to achieve functional levels of androgenic steroids in men and particularly in women, thus improving their health.

### SUMMARY OF THE INVENTION

Accordingly, the present invention provides a new method for improving the health of a person, especially a woman or man who has a low level of androgenic steroids, e.g. testosterone.

In the invention, the drug delivery method includes administering a testosterone-containing moiety, in an amount sufficient to provide a therapeutic effect of testosterone to a patient in need, by topical application of said testosterone-containing moiety to the eye of said patient. Examples of specific testosterone-containing moieties which may be utilized in the method of this invention include but are not limited to: testosterone, methyltestosterone, 17α-methylnortestosterone, dihydrotestosterone, testosterone propionate, testosterone cypionate, testosterone phenylacetate, testosterone enanthate, testosterone acetate, testosterone buciclate, testosterone heptanoate, testosterone decanoate, testosterone caprate, testosterone isocaprate, and combinations thereof.

The amount of testosterone-containing moiety to be administered may be measured according to several different parameters. In one aspect, the amount of testosterone-containing moiety administered may be an amount sufficient to achieve a therapeutic effect equivalent to a total testosterone serum level of from about 15 to about 1000 ng/dL.

The administration of the testosterone-containing moiety employed in accordance with the method of the present invention comprises topical administration of a liquid composition of said testosterone-containing moiety to the eye of a patient in need of treatment. In particular, the liquid composition including said testosterone-containing moiety is administered in the form of eye drops.

There are many indicators of the improved health which may occur as a result of the method of the present invention. Of particular note are the restoration, enhancement, improvement, or prevention of characteristics such as: sexual desire, frequency of sexual activity, stimulation of sexual organs, ability to achieve orgasm, pleasure in sexual activity, vital energy, sense of well-being, mood and sense of emotional well being, cognitive abilities, muscle mass and function, body composition, bone mineral density, skin and hair condition, pubic hair, urogenital atrophy, vaginal dryness, dry eyes, health in autoimmune conditions, vasomotor instability, breast tenderness, symptoms of premenstrual syndrome, and combination thereof.

### BRIEF DESCRIPTION OF THE DRAWINGS

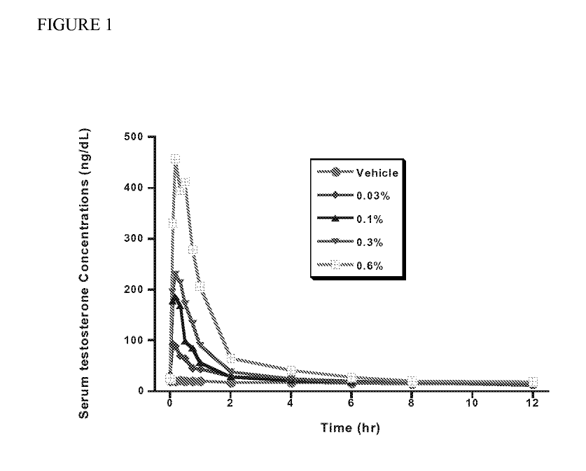

In FIG. 1 there is shown median Serum testosterone concentration-time profiles on day 13 from healthy postmenopausal women receiving testosterone ophthalmic solution 0.03%, 0.1%, 0.3%, 0.6% or vehicle ophthalmic solution, in each eye twice daily for 14 days.

### DETAILED DESCRIPTION OF THE INVENTION

A. Definitions

In describing and claiming the present invention, the following terminology will be used.

“Testosterone” refers to the compound having the IUPAC names (17β)-17-Hydroxyandrost-4-en-3-one, and Δ⁴ -androsten-17β-ol-3-one, as well as their isomers.

“Testosterone-containing moiety” refers to any product which can be used to deliver testosterone through the eye to the patient's blood stream. For example, compounds which are derivatives and/or analogues of testosterone are suitable for use in the method of this invention and include, but are not limited to: testosterone, methyltestosterone, androstenedione, 17α-methylnortestosterone, dihydrotestosterone, danazol, mesterolone, testosterone propionate, testosterone cypionate, testosterone phenylacetate, and testosterone enanthate, testosterone acetate, testosterone buciclate, testosterone heptanoate, testosterone decanoate, testosterone caprate, testosterone isocaprate, as well as esters, derivatives, prodrugs, and isomers thereof.

For the purpose of the present invention, “administration,” and “administering” refer to the manner in which a drug is presented to a subject. Administration is accomplished by topical application of a liquid composition including testosterone-containing moiety to the eye of the patient.

“Topical formulation” means a composition in which the drug may be placed for direct application to the eye and from which an effective amount of drug is released. Examples of topical formulations include but are not limited to ointments, creams, gels, patches, sprays, and pastes.

Ocular delivery system refers to a liquid type of delivery device which is used to deliver defined doses of a substance, over a specific application period.

The terms “formulation” and “composition” are used interchangeably herein. The terms “pharmaceutical” and “drug” are also used interchangeably to refer to a pharmacologically active substance or composition. These terms of art are well-known in the pharmaceutical and medicinal arts.

“Total serum level”, “total blood level”, and “endogenous serum level,” refer to the total serum levels of testosterone, including both protein-bound and free testosterone. Certain proteins such as albumin bind testosterone with a low affinity such that these sex hormones are functional (bioavailable) (i.e., produce its known or intended biological effect).

Thus, the term “total testosterone serum level” refers to the sum of: (1) free testosterone; and (2) testosterone which is weakly bound to serum proteins.

The term “protein-bound” includes all types of protein bindings.

“Man” refers to a human male who benefits from an androgen supplementation in any way.

“Woman” refers to a human female who benefits from an androgen supplementation in any way.

“Improving health” refers to reducing, improving, or preventing the incidence and/or intensity of symptoms associated with testosterone deficiency. Examples of such symptoms include but are not limited to: sexual dysfunction, which can manifest in loss of sexual desire, decreased sensitivity to sexual stimulation, decreased arousability and capacity for orgasm, diminished vital energy, depressed mood, diminished sense of well-being, increased shyness, loss of muscle mass and function, unfavorable body composition, i.e., lean to fat mass ratio, thinning and loss of pubic hair, urogenital atrophy, dry and brittle scalp hair, dry skin, decreased cognitive abilities, dry eyes, autoimmune phenomena, and a combination thereof.

“Effective amount” refers to an amount of a substance which is sufficient to achieve its intended purpose or effect. Various biological factors may affect the ability of a delivered substance to perform its intended task. Therefore, an “effective amount” may be dependent on such biological factors. A dosage would be considered to be an “effective amount” as long as it achieves its desired effect. Determination of an “effective amount” is well within the ordinary skill in the art.

Many evaluations may be employed for measuring the achievement of desired effects in the case of testosterone delivery, which are well known in the art. Such evaluations may be performed by a physician, or other qualified medical personnel, and may include physical examination, blood tests, etc.

“Therapeutic effect” refers to a desired result which is achieved to some degree. In the context of testosterone supplementation as presented in the present patent application, a number of desired results are referred to as “improving health.” In one aspect, therapeutic effects may be achieved by delivering an “effective amount” of a substance capable of achieving the desired result to a selected degree. While the achievement of therapeutic effects may be measured by a physician or other qualified medical personnel using evaluations known in the art, it is recognized that individual variation and response to treatments may make the achievement of therapeutic effects a subjective decision.

B. The Invention

Recent approvals of testosterone gels and testosterone buccal delivery system have shown that androgens, and particularly testosterone, contribute substantially to men's health and well-being. Recent research has shown that androgens, and particularly testosterone, contribute substantially also to a woman's health and well-being. Ebert, et al., U.S. Pat. No. 5,460,820, in one aspect, teaches a composition and method for administering testosterone transdermally via a patch delivery system. These compositions and methods maintain total testosterone serum blood levels in a “physiological range” of between about 15 to 80 ng/dL by means of transdermally administering about 50 to 500 mcg/day of testosterone from a testosterone matrix. It is recognized that non-oral delivery of androgens is safer to the liver and provides more sustained delivery than oral routes since the first pass metabolism effects are bypassed.

A clinical study entitled “A Single-Center, Double Masked, Parallel, Dose-Ranging, Vehicle Controlled Study Evaluating the Safety and Pharmacokinetics of Testosterone (0.03%, 0.1%, 0.3%, and 0.6%) Ophthalmic Solutions for up to 14 Days in Postmenopausal Women” was conducted to determine the effectiveness of the ocular administration of testosterone. This study was a randomized, single-center, double-masked, parallel-group study of five treatment groups, with each subject acting as her own baseline control for serum testosterone concentrations. Subjects received testosterone ophthalmic solution 0.03%, 0.1%, 0.3%, 0.6% or vehicle ophthalmic solution in each eye twice daily for 14 days. Each subject had blood withdrawn for the measurement of serum testosterone concentrations. On day 13, blood samples were taken prior to the morning dose (time 0), and at 5, 10, 20, 30, and 45 minutes, 1, 2, 4, 6, 8, and 12 hours post-dose. On day −1, blood samples were taken at the same time as day 13 to evaluate the baseline serum testosterone levels. On days −6, 7, 14, and 27, one blood sample was taken at the same time as day 13 predose draw to evaluate the trough serum testosterone levels (there were no dosing on days −6, −1, and 27). Maximal observed serum testosterone concentration (Cₘₐₓ), time corresponding to Cₘₐₓ (Tₘₐₓ), and area under the serum testosterone concentration-time curve from 0 to 12 hours (AUC₀₋₁₂ or AUC₀₋₁₂ₕᵣ) were calculated from both days −1 and 13 serum testosterone concentration data, the differences of Cₘₐₓ and AUC₀₋₁₂ₕᵣ between day 13 and day −1 were also calculated. Serum testosterone concentrations were assayed using a validated double antibody radioimmunoassay method with a lower limit of quantitation (LLOQ) at 29 pg/mL or 2.9 ng/dL.

The study found the following: After topical applications of testosterone ophthalmic solution 0.03%, 0.1%, 0.3%, 0.6% or vehicle ophthalmic solution in each eye twice daily for 14 days in healthy post-menopausal women:

1. The serum testosterone concentration reached the peak rapidly after dosing of testosterone ophthalmic solution, then returned quickly to the baseline level in 12 hours. Both Cₘₐₓ and AUC₀₋₁₂ₕᵣ appeared to have increased with an increase in dose.

2. The median Cₘₐₓ values of serum testosterone concentrations for the testosterone 0.03%, 0.1%, 0.3%, and 0.6% treatment groups were increased to much higher than the endogenous baseline level of about 20 ng/dL, and were 93.5, 193, 237, and 457 ng/dL, respectively.

(See FIG. 1 for the results of this study.)

The rate of testosterone production in ovulating women is reported to be about 0.4±0.1 mg/day (Vierhapper H, Nowotny P and Waldhausl W. “Determination of testosterone production rates in men and women using stable isotope/dilution and mass spectrometry.” J. Clin. Endocrinol. Metab. 1997; 82: 1492-1496.) or 0.25 mg/day (Goodman and Gilman's, 1996). The total daily doses following topical dosing of 50 μL of 0.03%, 0.1%, 0.3%, and 0.6% testosterone formulation BID to both eyes are 0.06, 0.2, 0.6, and 1.2 mg, respectively. Assuming the rate of testosterone production in post-menopausal women in current study is 0.125 mg/day, since the average endogenous serum testosterone level observed in this study is about 20 ng/mL, average increases of 3.8, 15, 24, and 49 ng/dL are equivalent to an absorbed daily dose of 0.024, 0.094, 0.15, and 0.31 mg, respectively.

Another clinical study entitled “A Multi-Center, Parallel, Randomized, Double-Masked, Vehicle-Controlled, Dose-Ranging Study to Evaluate the Safety, Tolerability and Efficacy of Testosterone 0.01%, 0.1%, and 0.3% Ophthalmic Solutions Administered Twice Daily for 16 weeks in Patients with Keratoconjunctivitis Sicca (KCS)” was conducted to determine the effectiveness of the ocular administration of testosterone in both women and men. This study was a randomized, multi-center, double-masked, parallel-group study of three treatment groups and one vehicle group. Patients were randomized to either 0.01%, 0.1%, 0.3% testosterone or vehicle of 0.3% testosterone ophthalmic solution. In the treatment phase, patients were instructed to instill one drop of the randomly assigned masked treatment twice daily (in the morning upon waking and before bedtime) for 16 weeks. Data from 135 patients (100 females and 35 males) were included in the pharmacokinetic data analysis. A single blood sample (20 mL) was taken from each patient pre-dose and at 10 minutes post dose on day 0, weeks 1, 4, 8, 12, and 16 for the determination of serum testosterone, dihydrotestosterone, 3-alpha-androstanediol glucuronide and sex hormone binding globulin concentrations. Serum concentrations of testosterone and dihydrotestosterone were determined by a validated gas chromatography-mass spectrometry (GC-MS) method with a quantitation range for DHT of 0.1 to 100 ng/dL and for testosterone of 1.0 to 1000 ng/dL. Serum concentrations of 3-alpha-androstanediol-glucuronide were quantitated by a validated radioimmunoassay method with a range of quantitation from 0.800 to 100 ng/mL, and serum concentrations of sex hormone binding globulin were determined using a validated two-site immunoradiometric assay (IRMA) method with a range of quantitation of 5 to 320 nmol/L for a 1:100 diluted sample.

The study found the following: after topical applications of testosterone ophthalmic solution 0.01%, 0.1%, 0.3%, or vehicle ophthalmic solution in each eye twice daily for 16 weeks in patients with keratoconjunctivitis sicca:
1. Serum testosterone concentrations for the testosterone 0.01%, 0.1%, and 0.3% treatment groups at 10 minutes after dosing in female patients resulted in a concentration dependent increase from the endogenous baseline level of approximately 20 ng/dL to 35, 200, and 350 ng/dL, respectively.
2. At 10 minutes after dosing in male patients, serum testosterone concentrations for the testosterone 0.1% and 0.3% treatment groups increased 13 and 36% respectively over the endogenous baseline.
3. Serum 3-alpha-androstanediol glucuronide and sex hormone binding globulin concentrations appeared to be unaffected by ocular administration of testosterone in both female and male patients over 16 weeks of dosing period.
4. The serum pharmacokinetic data did not indicate a safety concern during the 4 month dosing period at all three testosterone 0.01%, 0.1%, and 0.3% concentration dose levels.

Therefore, it is concluded from this study that testosterone can be effectively delivered ophthalmically to either a woman or a man for hormone replacement therapy. Physiologic amounts of testosterone can be delivered ophthalmically producing circulating testosterone levels that approximate normal levels (e.g., 15-85 ng/dL) seen in healthy women. Physiologic amounts of testosterone can also be delivered ophthalmically producing circulating testosterone levels that approximate normal levels (e.g., 300-1000 ng/dL) seen in healthy men.

The kinetic profile found in ophthalmical delivering testosterone as an eye drop is unique as compared to any other testosterone delivery systems. Peak serum testosterone concentration is reached 5 to 10 minutes after ophthalmical dosing, about 90% of the peak level is gone in 2 hours. This unique feature ensures a rapid onset of drug action, and at the same time minimizes the down regulation of endogenous androgen production, as well as significantly reduces the probability of any androgen related adverse effects.

C. The Various Aspects

In one aspect of the present invention, a testosterone-containing moiety may be administered at a dosage sufficient to achieve a therapeutic effect equivalent to a total testosterone serum level of from about 15 to about 1000 ng/dL. In another aspect of the invention a testosterone-containing moiety may be administered at a dosage sufficient to achieve a therapeutic effect equivalent to a total testosterone serum level of from about 85 to about 1000 ng/dL. In a further aspect of the invention, a testosterone-containing moiety may be administered at a dosage sufficient to achieve a therapeutic effect equivalent to a total testosterone serum level of from about 100 to about 1000 ng/dL.

The delivery for a testosterone-containing moiety dose is topical administration through the eye of a patient. Topical formulations may include an effective amount of a testosterone-containing moiety may be incorporated into a pharmaceutically acceptable carrier. Various carriers will be suitable based on the type of delivery formulation desired. In general, the liquid, testosterone-containing moiety compositions of the invention may comprise from 0.001 to 5% by weight testosterone, e.g. from 0.01 to 1% or more preferably from 0.3 to 0.6%, by weight testosterone.

The need for supplementing sex hormones such as testosterone should be determined by a physician or other health care professional based on monitoring signs and symptoms of sex hormone deficiency or based on need for pharmacological intervention of conditions that are responsive to hormonal therapy.

Symptoms of subfunctional levels of testosterone, might include, but not be limited to: sexual dysfunction, which can manifest in loss of sexual desire, decreased sensitivity to sexual stimulation, decreased arousability and capacity for orgasm; diminished vital energy; depressed mood; diminished sense of well-being; increased shyness; loss of muscle mass and function; unfavorable body composition, i.e., lean to fat mass ratio; thinning and loss of pubic hair; urogenital atrophy; dry and brittle scalp hair; dry skin; decreased cognitive abilities; dry eyes; autoimmune phenomena or exacerbation thereof, and a combination thereof.

1. A method of improving sexual health and activity, maximizing muscle mass and function, inhibiting bone loss, improving cardiovascular and coronary health, decreasing breast tenderness and vasomotor instability, modulating immune function, enhancing cognitive abilities, improving urogential health, reducing estrogen supplementation related side effects, and providing direct neuroprotective effects in a woman or man comprising non-orally administering a testosterone-containing moiety to said woman or man by topically applying a liquid composition including said testosterone-containing moiety to the eye of said woman or man in an amount sufficient to provide a therapeutic effect.

2. The method of claim 1, wherein said testosterone is administered as a member selected from the group consisting of: testosterone, methyltestosterone, 17α-methylnortestosterone, dihydrotestosterone, testosterone propionate, testosterone cypionate, testosterone phenylacetate, testosterone enanthate, testosterone acetate, testosterone buciclate, testosterone heptanoate, testosterone decanoate, testosterone caprate, testosterone isocaprate, and isomers thereof, and a combination thereof.

3. The method of claim 2, wherein said testosterone is administered as a member selected from the group consisting of: testosterone, dihydrotestosterone, methyltestosterone, and isomers thereof, and a combination thereof.

4. The method of claim 1, wherein said testosterone is administered in a dosage sufficient to achieve a therapeutic effect equivalent to a total testosterone serum level of from about 15 to about 1000 ng/dL.

5. A method of improving sexual health and activity, maximizing muscle mass and function, inhibiting bone loss, improving cardiovascular and coronary health, decreasing breast tenderness and vasomotor instability, modulating immune function, enhancing cognitive abilities, improving urogential health, reducing estrogen supplementation related side effects, and providing direct neuroprotective effects in a woman or man comprising non-orally administering a testosterone-containing moiety to said woman or man by topically applying a liquid composition including said testosterone-containing moiety to the eye of said woman or man to provide a rapid onset of therapeutic effect, and at the same time [minimizes] minimizing the down regulation of endogenous androgen production, as well as significantly reducing the probability of any androgen related adverse effects.

6. A method of improving sexual health and activity, maximizing muscle mass and function, inhibiting bone loss, improving cardiovascular and coronary health, decreasing breast tenderness and vasomotor instability, modulating immune fiunction, enhancing cognitive abilities, improving urogential health, reducing estrogen supplementation related side effects, and providing direct neuroprotective effects in a woman comprising non-orally administering testosterone to said woman or man by topically applying a liquid composition including testosterone to the eye of said woman or in an amount sufficient to provide a daily dose of from 0.06 to 1.2 mg of testosterone to said woman to thereby provide a rapid onset of therapeutic effect, and at the same time minimize the down regulation of endogenous androgen production, as well as significantly reduce the probability of any androgen related adverse effects.

7. The method of claim 6 wherein said liquid composition comprises from 0.03 to 0.6% testosterone.

8. The method of claim 7 wherein the serum level of testosterone of said woman is raised to a peak serum level of from 93.5 to 457 ng/dL within 5 to 10 minutes from ophthalmic dosing.

9. The method of claim 8 wherein said peak serum level of testosterone is reduced by 90% within two hours of administration.

10. A method of improving sexual health and activity, maximizing muscle mass and function, inhibiting bone loss, improving cardiovascular and coronary health, decreasing breast tenderness and vasomotor instability, modulating immune function, enhancing cognitive abilities, improving urogential health, reducing estrogen supplementation related side effects, and providing direct neuroprotective effects in a woman comprising non-orally administering testosterone to said woman by topically applying a composition comprising a solution including from 0.03% to 0.6% testosterone to the eye of said woman in an amount sufficient to provide a serum testosterone concentration of from 93.5 to 457 ng/dL a therapeutic effect.

11. The method of claim 6 wherein said composition is administered twice daily.

12. The method of claim 11 wherein the peak serum concentration of testosterone is reached in 5 to 10 minutes from topical application.

13. The method of claim 12 wherein said peak serum concentration is reduced by 90% in 2 hours.

14. A method of improving sexual health and activity, maximizing muscle mass and function, inhibiting bone loss, improving cardiovascular and coronary health, decreasing breast tenderness and vasomotor instability, modulating immune function, enhancing cognitive abilities, improving urogential health, reducing estrogen supplementation related side effects, and providing direct neuroprotective effects in a woman comprising non-orally administering testosterone to said woman by topically applying 50 ul of a composition comprising a solution including from 0.03% to 0.6% testosterone, twice daily, to the eye of said woman to provide a peak serum testosterone concentration of from 93.5 to 457 mg/dL, wherein said peak serum testosterone concentration is reached in 5 to 10 minutes from topical application and is reduced by 90% in 2 hours thereby provide a rapid onset of therapeutic effect, and at the same time minimize the down regulation of endogenous androgen production, as well as significantly reduce the probability of any androgen related adverse effects.
